(19) United States
(12) Patent Application Publication
(10) Pub. No.: US 2026/0209241 A1
(43) Pub. Date: Jul. 23, 2026

(54) PROCESS FOR THE PREPARATION OF BENZGALANTAMINE OR A PHARMACEUTICALLY ACCEPTABLE SALT THEREOF

(71) Applicant: Zydus Lifesciences Limited, Gandhinagar (IN)

(72) Inventors: Kumar Kamlesh Singh, Gandhinagar (IN); Santosh Devidas Diwakar, Gandhinagar (IN); Sumer Singh Chundawat, Gandhinagar (IN); Dipak Bhatu Patil, Gandhinagar (IN); Rajesh Pandya, Gandhinagar (IN)

(21) Appl. No.: 19/432,458

(22) Filed: Dec. 24, 2025

(30) Foreign Application Priority Data
Dec. 26, 2024 (IN) 202421103154

(51) Int. Cl. C07D 491/06; A61K 31/55

(52) U.S. Cl. CPC C07D 491/06; A61K 31/55

(57) ABSTRACT

The present invention relates to a process for the preparation of benzgalantamine, or a pharmaceutically acceptable salt, or a hydrate thereof. The present invention also relates to benzgalantamine or a pharmaceutically acceptable salt, or a hydrate thereof substantially free of one or more impurities of compounds of Formulae A, B, C, or D as described in the specification. The process of the present invention further provides benzgalantamine or a pharmaceutically acceptable salt, or a hydrate thereof, substantially free of nitrosamine impurities of compounds of Formulae E and F. The present invention also provides a crystalline form of benzgalantamine and a process for the preparation thereof.

### CROSS REFERENCE TO RELATED APPLICATION

This application claims priority to Indian Application No. 202421103154, filed Dec. 26, 2024, which is hereby incorporated by reference in its entirety.

### FIELD OF THE INVENTION

The present invention relates to a process for the preparation of benzgalantamine, or a pharmaceutically acceptable salt, or a hydrate thereof. The present invention also relates to benzgalantamine or a pharmaceutically acceptable salt, or a hydrate thereof substantially free of one or more impurities of compounds of Formulae A, B, C, or D as described herein in the specification. The process of the present invention further provides benzgalantamine or a pharmaceutically acceptable salt, or a hydrate thereof, substantially free of nitrosamine impurities of compounds of Formulae E and F. The present invention also provides a crystalline form of benzgalantamine and a process for the preparation thereof.

### BACKGROUND OF THE INVENTION

The following discussion of the prior art is intended to present the invention in an appropriate technical context and allow its significance to be properly appreciated. Unless clearly indicated to the contrary, however, reference to any prior art in this specification should be construed as an admission that such art is widely known or forms part of common general knowledge in the field.

Benzgalantamine chemically known as (4aS,6R,8aS)-4a,5,9,10,11,12-hexahydro-3-methoxy-11-methyl-6H-benzofuro[3a,3,2-ef][2]benzazepin-6-benzoate and structurally represented as the compound of Formula I below:

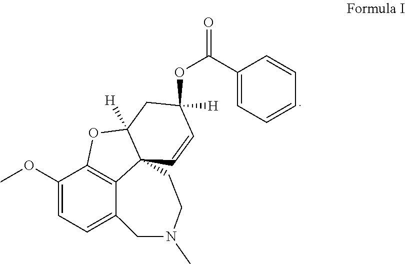 [SMILES: COc1ccc2c3c1O[C@H]1C[C@@H](OC(=O)c4ccccc4)C=C[C@@]31CCN(C)C2]

is a prodrug of galantamine, a cholinesterase inhibitor. It was approved by USFDA under the brand name ZUNVEYL®. The ZUNVEYL tablets contain benzgalantamine in the form of gluconate salt of benzgalantamine. The benzgalantamine gluconate is represented by the following structural Formula II:

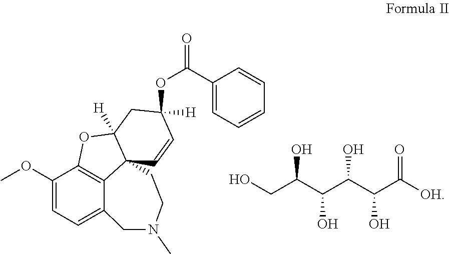 [SMILES: COc1ccc2c3c1O[C@H]1C[C@@H](OC(=O)c4ccccc4)C=C[C@@]31CCN(C)C2 ; O=C(O)[C@H](O)[C@@H](O)[C@H](O)[C@H](O)CO]

U.S. Pat. No. 9,763,953 discloses benzgalantamine and a general process for its preparation.

U.S. Pat. No. 11,077,119 discloses gluconate salt of benzgalantamine and a process for its preparation.

U.S. Pat. No. 11,795,176 discloses crystalline forms of benzgalantamine gluconate.

Journal article: Molecules 2021, 26, 6023, discloses 6-O-benzoylgalanthamine as a white amorphous solid.

Still there is a need to develop a process that is economically viable and industrially scalable for preparing benzgalantamine or a pharmaceutically acceptable salt thereof.

### SUMMARY OF THE INVENTION

In one general aspect, the present invention provides a process for the preparation of benzgalantamine of Formula I,

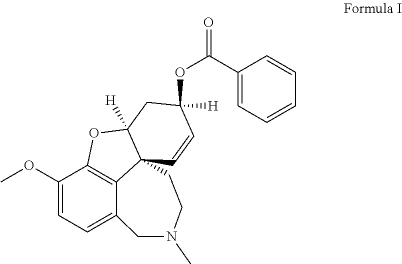 [SMILES: COc1ccc2c3c1O[C@H]1C[C@@H](OC(=O)c4ccccc4)C=C[C@@]31CCN(C)C2]

or a pharmaceutically acceptable salt, or a hydrate thereof, the process comprising:
(a) reacting galantamine or a salt thereof with benzoyl chloride in the presence of one or more bases in one or more solvents to obtain benzgalantamine of Formula I, wherein the solvents are selected from one or more of acetonitrile, acetone, ethylacetate, toluene, N,N-dimethylformamide, tetrahydrofuran, methyl ethyl ketone, 1,4-dioxane, and methyl isobutyl ketone; and
(b) optionally, converting the benzgalantamine of Formula I obtained at step (a) to its pharmaceutically acceptable salt, or hydrate thereof.

In another general aspect, the present invention provides a compound selected from a compound of Formula A, or a compound of Formula C,

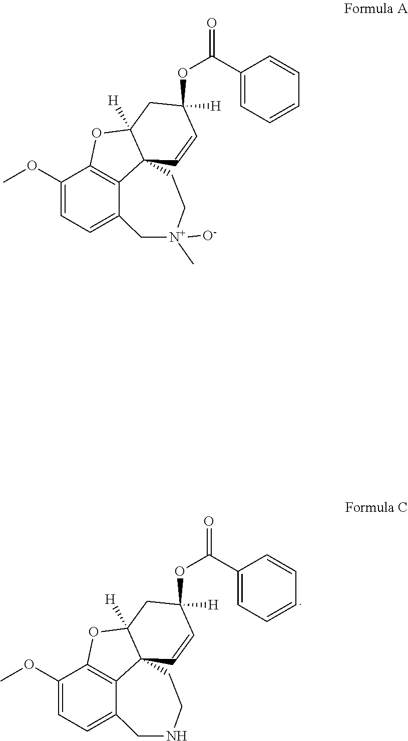

In another general aspect, the present invention provides the impurities selected from compound of Formula A, or a compound of Formula C as a “reference marker” and/or “reference standard” in methods for determining the identity and/or the amount of said impurities in a sample of benzgalantamine or a pharmaceutically acceptable salt thereof.

In another general aspect, the present invention provides a composition comprising benzgalantamine of Formula I or a pharmaceutically acceptable salt, or a hydrate thereof, having a purity of about 99% or more, as measured by area percentage of high performance liquid chromatography (HPLC), and one or more impurities of compounds of Formulae A, B, C, and D, wherein each of the compounds of Formulae A, B, C, or D is present in an amount of about 0.15% or less, by weight, relative to benzgalantamine of Formula I or a pharmaceutically acceptable salt, or a hydrate thereof, when measured by HPLC,

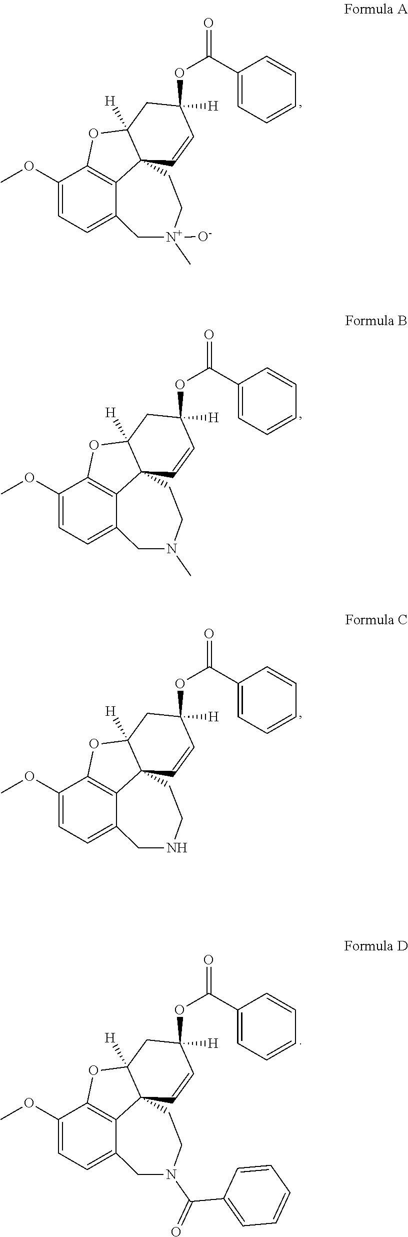

In another general aspect, the present invention provides a composition comprising benzgalantamine of Formula I or a pharmaceutically acceptable salt, or a hydrate thereof, having a purity of about 99% or more, as measured by area percentage of HPLC, and one or more impurities of compounds of Formulae A, B, C, and D, wherein each of the compounds of Formulae A, B, C, or D when present, is in a detectable amount of about 0.15% or less by weight, relative to benzgalantamine of Formula I or a pharmaceutically acceptable salt or a hydrate thereof, when measured by HPLC.

In another general aspect, the present invention provides a benzgalantamine of Formula I or a pharmaceutically acceptable salt, or a hydrate thereof substantially free of one or more impurities of compounds of Formulae A, B, C, or D.

In another general aspect, the present invention provides crystalline benzgalantamine of Formula I.

In another general aspect, the present invention provides a crystalline form of benzgalantamine of Formula I, characterized by X-ray powder diffraction pattern having peaks expressed in 20 at 8.1°, 16.3°, 17.7°, 19.9°, and 20.8°±0.2°, when measured using CuKα source.

In another general aspect, the present invention provides a process for the preparation of a crystalline form of benzgalantamine of Formula I, characterized by X-ray powder diffraction pattern having peaks expressed in 20 at 8.1°, 16.3°, 17.7°, 19.9°, and 20.8°±0.2°, when measured using CuKα source, the process comprising:
(i) adding water to a reaction mass comprising benzgalantamine and acetonitrile, and
(ii) obtaining the crystalline form of benzgalantamine.

The process of the present invention is a better process that is economically viable and industrially scalable for preparing benzgalantamine or a pharmaceutically acceptable salt or a hydrate thereof. Also, the present invention provides benzgalantamine or a pharmaceutically acceptable salt or a hydrate thereof with high purity and yield.

The process of the present invention provides benzgalantamine of Formula I or a pharmaceutically acceptable salt, or a hydrate thereof, substantially free of one or more impurities of compounds of Formulae A, B, C, or D. Further the process provides benzgalantamine of Formula I or a pharmaceutically acceptable salt, or a hydrate thereof, containing about 0.58 ppm or less of the nitrosamine impurity of compound of Formula E, and about 0.58 ppm or less of the nitrosamine impurity of compound of Formula F,

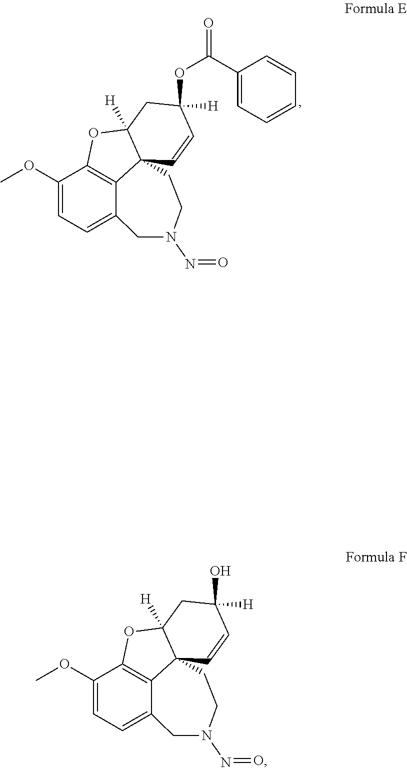 [SMILES: COc1ccc2c3c1O[C@H]1C[C@@H](OC(=O)c4ccccc4)C=C[C@@]31CCN(N=O)C2 ; COc1ccc2c3c1O[C@H]1C[C@@H](O)C=C[C@@]31CCN(N=O)C2]

as determined by an LCMS method.

### BRIEF DESCRIPTION OF DRAWINGS

FIG. 1. X-ray powder diffraction pattern of the crystalline form of benzgalantamine obtained in example 1.

FIG. 2. Differential Scanning calorimetry (DSC) of the crystalline form of benzgalantamine obtained in example 1.

### DETAILED DESCRIPTION OF THE INVENTION

The invention can further be understood in light of the description of the embodiments provided herein after. It is to be understood that the description, in no way, is intended to limit the scope of the invention to the expressly specified embodiments only. The equivalents and variants thereof or trivial modifications thereof which are apparently obvious to those skilled in the art, are also intended to be included within the scope of the present invention.

Detailed description of routine and conventional unit operations, which are easily understood by the skilled artisan, are not included herein. Such routine unit operations are to be construed as ordinarily understood and as routinely practiced by the person skilled in the field of the invention, unless otherwise specifically described.

The following definitions are used in connection with present application, unless it is indicated otherwise.

In general, the term “reacting” is used in their ordinary meaning as they are used in the field of the invention, unless defined specifically otherwise.

The term “obtaining” include but not specifically limited to decantation, precipitation, extraction, filtration, evaporation, lyophilisation, spray drying, crystallization, recrystallization, or chromatographic operations.

The term “optionally” is taken to mean that the event or circumstance described in the specification may or may not occur, and that the description includes instances where the event occurs and instances where it does not.

The term “pharmaceutically acceptable” means that which is useful in preparing a pharmaceutical composition that is generally non-toxic and is not biologically undesirable, and includes that which is acceptable for veterinary use and/or human pharmaceutical use.

The term “substantially free of one or more impurities of compounds of Formulae A, B, C or D” herein refers to benzgalantamine or a pharmaceutically acceptable salt or a hydrate thereof containing one or more impurities of compounds of Formulae A, B, C or D in an amount within the permissible ICH limits suitable for pharmaceutical preparations. For example, but not limited to about 0.15% or less, or to about 0.1% or less, or more particularly to about 0.05% or less, or most particularly not in detectable amount, by weight, relative to benzgalantamine of Formula I or a pharmaceutically acceptable salt or a hydrate thereof, when measured by HPLC, or absent.

The term “substantially free of nitrosamine impurities of Formula E and F” as used herein refers to said compound containing nitrosamine impurities of Formula E and F, each in an amount of about 0.58 ppm or less, as determined by an LCMS method. In particular, less than about 0.4 ppm, less than about 0.3 ppm, less than about 0.2 ppm, less than about 0.1 ppm, less than about 0.05 ppm, less than about 0.006 ppm, particularly not in detectable amount as determined by LCMS method, or absent.

The phrase “wherein each of the compounds of Formulae A, B, C, or D is present in an amount of about 0.15% or less” means each of the compound of Formula A, Formula B, Formula C, or Formula D is present in the composition in an amount of about 0.15% or less, or about 0.1% or less, or about 0.05% or less, or about 0.05% or less, or not in detectable amount, by weight, when measured by area percentage of HPLC, or absent.

Alternatively, the phrase “wherein each of the compounds of Formulae A, B, C, or D is present in an amount of about 0.15% or less” refers to each of the compounds of Formulae A, B, C, or D when present in the composition, is present in a detectable amount of about 0.15% or less, by weight, relative to benzgalantamine of Formula I or a pharmaceutically acceptable salt or a hydrate thereof, when measured by area percentage of HPLC.

The term “in a detectable amount”, refers to the level of impurity in the product, which is in the level of detection limit, when analyzed using the HPLC method.

The term “not in detectable amount” or “non-detectable amount”, refers to the level of impurity in the product, which is below the level of detection limit, when analyzed using the HPLC method/LCMS method.

The term “about” as used herein, is intended to mean approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 10%.

The term “composition” used herein means a physical mixture of two or more components.

The term “excipient(s)” or “pharmaceutically acceptable excipient(s)” refers to pharmacologically inactive substances that are added to a pharmaceutical preparation in addition to the active pharmaceutical ingredient. The pharmaceutically acceptable excipients may include one or more diluents, binders, disintegrants, lubricants, glidants, sweeteners/taste masking agents, colorants, flavours, film-forming agents, plasticizers and the like.

The pharmaceutical acceptable salts of benzgalantamine includes the acid addition salts such as those formed from gluconic acid, saccharic acid, lactic acid, citric acid, hydrochloric acid, hydrobromic acid, and the like.

The product(s) obtained may further be purified to obtain them in purer form. The product(s) obtained may further be dried additionally to achieve desired level of moisture and/or residual solvents.

The product(s) obtained may further be converted to any other physical forms thereof which includes but not specifically limited to polymorph(s), salt(s), solvate(s), hydrate(s), co-crystal(s) or solid dispersion(s); and crystalline or amorphous forms thereof.

The product(s) obtained may further be subjected to physical processing which includes, but not limited to, pressing, crushing, triturating, milling, or grinding to adjust the particle size of the product(s) to desired levels.

Thus, in one general aspect, the present invention provides a process for the preparation of benzgalantamine of Formula I, or a pharmaceutically acceptable salt, or a hydrate thereof, the process comprising:
(a) reacting galantamine or a salt thereof with benzoyl chloride in the presence of one or more bases in one or more solvents to obtain benzgalantamine of Formula I, wherein the solvent is selected from one or more of acetonitrile, acetone, ethylacetate, toluene, N,N-dimethylformamide, tetrahydrofuran, methyl ethyl ketone, 1,4-dioxane, and methyl isobutyl ketone; and
(b) optionally, converting the benzgalantamine of Formula I obtained at step (a) to its pharmaceutically acceptable salt, or a hydrate thereof.

In general, the base at step (a) is selected from one or more of triethylamine, N,N-diisopropylethylamine, pyridine, and 1,8-diazabicyclo(5.4.0)-undec-7-ene. In particular, the base is triethylamine.

In general, the reaction at step (a) is performed in the presence of an activating agent 4-dimethylaminopyridine (DMAP).

In general, the activating agent DMAP at step (a) is used in an amount of about 0.5 to about 1.5 mole equivalent relative to galantamine or salt thereof. In particular, DMAP is used in an amount of 1 mole equivalent relative to galantamine or salt thereof.

In an embodiment, the solvent at step (a) is acetonitrile.

In another embodiment, the reaction at step (a) is performed optionally in the presence of an anti-oxidant selected from butylated hydroxytoluene (BHT), butylated hydroxyanisole (BHA), sodium thiosulfate, and sodium metabisulfite. In particular, the anti-oxidant is butylated hydroxytoluene.

In another embodiment, the reaction at step (a) is performed in the presence of an anti-oxidant. A suitable anti-oxidant for the purpose is selected from butylated hydroxytoluene (BHT), butylated hydroxyanisole (BHA), sodium thiosulfate, and sodium metabisulfite. In particular, the anti-oxidant is butylated hydroxytoluene.

In general, the anti-oxidant at step (a) is used in an amount of about 0.1% w/w to about 0.2% w/w relative to galantamine or a salt thereof.

In general, the reaction at step (a) is performed at a temperature of about 0° C. to about 50° C. In particular, the reaction at step (a) is performed at a temperature of about 0° C. to about 20° C. More particularly, the reaction at step (a) is performed at a temperature of about 0° C. to about 15° C.

In general, the solvent at step (a) is used in an amount of about 2 volumes to about 10 volumes of the quantity of galantamine or a salt thereof. In particular, the solvent at step (a) is used in an amount of about 3 to about 5 volumes of the quantity of galantamine or a salt thereof.

In general, benzoyl chloride is used at step (a) in an amount of about 1 mole equivalent to about 1.5 mole equivalent relative to galantamine or a salt thereof. In particular, the benzoyl chloride is used in an amount of 1.1 mole equivalent relative to galantamine or a salt thereof.

In another embodiment, the galantamine used at step (a) is in the form of galantamine hydrobromide.

In another embodiment, the pharmaceutically acceptable salt of benzgalantamine is benzgalantamine gluconate.

In another embodiment, benzgalantamine is converted into gluconate salt of benzgalantamine as in step (b).

In general, benzgalantamine is converted into its pharmaceutically acceptable salt, or a hydrate thereof by treating the benzgalantamine with appropriate acid or lactone thereof. For example, benzgalantamine is converted into its gluconate salt comprising treating the benzgalantamine with D-gluconic acid delta lactone.

In general, benzgalantamine is converted into its pharmaceutically acceptable salt, or a hydrate thereof, by treating the benzgalantamine with appropriate acid or lactone thereof in one or more suitable solvents. The suitable solvent for the purpose is selected from ethanol, isopropanol, methanol, 1,4-dioxane, water, or mixtures thereof. In particular, the solvent used for this purpose is selected from ethanol, water, or a mixture thereof. In particular, the solvent used for this purpose is free of peroxide.

In general, the solvent used in the conversion of benzgalantamine into its pharmaceutically acceptable salt, or a hydrate thereof, is first treated with an anti-oxidant. The anti-oxidant for the purpose is selected from butylated hydroxytoluene (BHT), butylated hydroxyanisole (BHA), sodium thiosulfate, or sodium metabisulfite. The solvent is then purified by distillation and subsequently employed in the reaction.

In another general aspect, the present invention provides a benzgalantamine of Formula I or a pharmaceutically acceptable salt, or a hydrate thereof substantially free of one or more impurities of compounds of Formulae A, B, C, or D.

In another aspect, the present invention provides a benzgalantamine of Formula I or a pharmaceutically acceptable salt, or a hydrate thereof having a purity of 99% or more when measured by area percentage of HPLC and wherein
(i) the content of compound of Formula A is about 0.15% or less,
(ii) the content of compound of Formula B is about 0.15% or less,
(iii) the content of compound of Formula C is about 0.15% or less, or
(iv) the content of compound of Formula D is about 0.15% or less, by weight relative to benzgalantamine of Formula I or a pharmaceutically acceptable salt or a hydrate thereof, when measured by HPLC.

In another general aspect, the present invention provides a composition comprising benzgalantamine of Formula I or a pharmaceutically acceptable salt, or a hydrate thereof, having a purity of about 99% or more, as measured by area percentage of high performance liquid chromatography (HPLC), and one or more impurities of compounds of Formulae A, B, C, and D, wherein each of the compounds of Formulae A, B, C, or D is present in an amount of about 0.15% or less, by weight, relative to benzgalantamine of Formula I or a pharmaceutically acceptable salt, or a hydrate thereof, when measured by HPLC.

In another embodiment, the pharmaceutically acceptable salt of benzgalantamine present in the composition is benzgalantamine gluconate.

In another embodiment, the present invention provides a composition comprising benzgalantamine gluconate, or a hydrate thereof, having a purity of about 99% or more as measured by area percentage of high performance liquid chromatography (HPLC), and one or more impurities of compounds of Formulae A, B, C, and D, wherein each of the compounds of Formulae A, B, C, or D is present in an amount of about 0.15% or less by weight, relative to benzgalantamine gluconate, or a hydrate thereof, when measured by HPLC.

In another embodiment, the present invention provides a composition comprising benzgalantamine gluconate, or a hydrate thereof, having a purity of about 99% or more as measured by area percentage of HPLC, and having
(i) the compound of Formula A in an amount of about 0.15% or less,
(ii) the compound of Formula B in an amount of about 0.15% or less,
(iii) the compound of Formula C in an amount of about 0.15% or less, or
(iv) the compound of Formula D in an amount of about 0.15% or less, by weight relative to benzgalantamine gluconate or a hydrate thereof, when measured by HPLC.

In another embodiment, the present invention provides a composition comprising benzgalantamine of Formula I or a pharmaceutically acceptable salt, or a hydrate thereof, having a purity of about 99.5% or more, as measured by area percentage of HPLC.

In another embodiment, the present invention provides a composition comprising benzgalantamine of Formula I or a pharmaceutically acceptable salt, or a hydrate thereof, having a purity of about 99.8% or more, as measured by area percentage of HPLC.

In another embodiment, benzgalantamine of Formula I, or a pharmaceutically acceptable salt, or a hydrate thereof, is having a purity of about 99.9%, or more, as measured by area percentage of HPLC.

In another embodiment, benzgalantamine of Formula I, or a pharmaceutically acceptable salt, or a hydrate thereof, is having a purity of about 100.0%, as measured by area percentage of HPLC.

In another embodiment, each of the compounds of Formulae A, B, C, or D, is present in the composition in an amount of about 0.00% to about 0.15% by weight, relative to benzgalantamine of Formula I or a pharmaceutically acceptable salt, or a hydrate thereof, when measured by HPLC.

In another embodiment, each of the compounds of Formulae A, B, C, or D, is present in the composition in an amount of about 0.001% to about 0.15% by weight, relative to benzgalantamine of Formula I or a pharmaceutically acceptable salt, or a hydrate thereof, when measured by HPLC.

In another embodiment, each of the compounds of Formulae A, B, C, or D, is present in the composition in an amount of about 0.001% to about 0.15% by weight, relative to benzgalantamine of Formula I or a pharmaceutically acceptable salt, or a hydrate thereof, when measured by HPLC, for example in an amount of about 0.002% to about 0.15%, about 0.003% to about 0.15%, about 0.004% to about 0.15%, about 0.005% to about 0.15%, about 0.01% to about 0.15%, about 0.001% to about 0.1%, about 0.002% to about 0.1%, about 0.003% to about 0.1%, about 0.004% to about 0.1%, about 0.005% to about 0.1%, or about 0.01% to about 0.1%, by weight, relative to benzgalantamine of Formula I or a pharmaceutically acceptable salt, or a hydrate thereof, when measured by HPLC.

In another embodiment, each of the compounds of Formulae A, B, C, or D, is present in the composition in an amount of about 0.003% to about 0.15% by weight, relative to benzgalantamine of Formula I or a pharmaceutically acceptable salt, or a hydrate thereof, when measured by HPLC.

In another embodiment, each of the compounds of Formulae A, B, C, or D, is present in the composition in an amount of about 0.01% to about 0.15% by weight, relative to benzgalantamine of Formula I or a pharmaceutically acceptable salt, or a hydrate thereof, when measured by HPLC.

In another embodiment, each of the compounds of Formulae A, B, C, or D, is present in the composition in an amount of about 0.01% to about 0.1% by weight, relative to benzgalantamine of Formula I or a pharmaceutically acceptable salt, or a hydrate thereof, when measured by HPLC.

In another aspect, the present invention provides a composition comprising benzgalantamine of Formula I or a pharmaceutically acceptable salt, or a hydrate thereof, having a purity of about 99% or more, as measured by area percentage of high performance liquid chromatography (HPLC), and a compound of Formula A present in the composition in an amount of about 0.15% or less by weight, relative to benzgalantamine of Formula I or a pharmaceutically acceptable salt or a hydrate thereof, when measured by HPLC.

In another embodiment, the compound of Formula A is present in the composition in an amount of about 0.00% to about 0.15% by weight, relative to benzgalantamine of Formula I or a pharmaceutically acceptable salt, or a hydrate thereof, when measured by HPLC. For example, in an amount of about 0.001% to about 0.15%, 0.002% to about 0.15%, about 0.003% to about 0.15%, about 0.004% to about 0.15%, about 0.005% to about 0.15%, about 0.01% to about 0.15%, about 0.001% to about 0.1%, about 0.002% to about 0.1%, about 0.003% to about 0.1%, about 0.004% to about 0.1%, about 0.005% to about 0.1%, or about 0.01% to about 0.1%, by weight, relative to benzgalantamine of Formula I or a pharmaceutically acceptable salt, or a hydrate thereof, when measured by HPLC.

In another aspect, the present invention provides a composition comprising benzgalantamine of Formula I or a pharmaceutically acceptable salt, or a hydrate thereof, having a purity of about 99% or more, as measured by area percentage of high performance liquid chromatography (HPLC), and a compound of Formula B present in the composition in an amount of about 0.15% or less by weight, relative to benzgalantamine of Formula I or a pharmaceutically acceptable salt, or a hydrate thereof, when measured by HPLC.

In another embodiment, the compound of Formula B is present in the composition in an amount of about 0.00% to about 0.15% by weight, relative to benzgalantamine of Formula I or a pharmaceutically acceptable salt, or a hydrate thereof, when measured by HPLC. For example, in an amount of about 0.001% to about 0.15%, 0.002% to about 0.15%, about 0.003% to about 0.15%, about 0.004% to about 0.15%, about 0.005% to about 0.15%, about 0.01% to about 0.15%, about 0.001% to about 0.1%, about 0.002% to about 0.1%, about 0.003% to about 0.1%, about 0.004% to about 0.1%, about 0.005% to about 0.1%, or about 0.01% to about 0.1%, by weight relative to benzgalantamine of Formula I or a pharmaceutically acceptable salt, or a hydrate thereof, when measured by HPLC.

In another aspect, the present invention provides a composition comprising benzgalantamine of Formula I or a pharmaceutically acceptable salt, or a hydrate thereof, having a purity of about 99% or more, as measured by area percentage of high performance liquid chromatography (HPLC), and a compound of Formula C present in the composition in an amount of about 0.15% or less by weight, relative to benzgalantamine of Formula I or a pharmaceutically acceptable salt, or a hydrate thereof, when measured by HPLC.

In another embodiment, the compound of Formula C is present in the composition in an amount of about 0.00% to about 0.15% by weight, relative to benzgalantamine of Formula I or a pharmaceutically acceptable salt, or a hydrate thereof, when measured by HPLC. For example, in an amount of about 0.001% to about 0.15%, 0.002% to about 0.15%, about 0.003% to about 0.15%, about 0.004% to about 0.15%, about 0.005% to about 0.15%, about 0.01% to about 0.15%, about 0.001% to about 0.1%, about 0.002% to about 0.1%, about 0.003% to about 0.1%, about 0.004% to about 0.1%, about 0.005% to about 0.1%, or about 0.01% to about 0.1%, by weight relative to benzgalantamine of Formula I or a pharmaceutically acceptable salt, or a hydrate thereof, when measured by HPLC.

In another aspect, the present invention provides a composition comprising benzgalantamine of Formula I or a pharmaceutically acceptable salt, or a hydrate thereof, having a purity of about 99% or more as measured by area percentage of high performance liquid chromatography (HPLC), and a compound of Formula D present in the composition in an amount of about 0.15% or less by weight, relative to benzgalantamine of Formula I or a pharmaceutically acceptable salt, or a hydrate thereof, when measured by HPLC.

In another embodiment, the compound of Formula D is present in the composition in an amount of about 0.00% to about 0.15% by weight, relative to benzgalantamine of Formula I or a pharmaceutically acceptable salt, or a hydrate thereof, when measured by HPLC. For example, in an amount of about 0.001% to about 0.15%, 0.002% to about 0.15%, about 0.003% to about 0.15%, about 0.004% to about 0.15%, about 0.005% to about 0.15%, about 0.01% to about 0.15%, about 0.001% to about 0.1%, about 0.002% to about 0.1%, about 0.003% to about 0.1%, about 0.004% to about 0.1%, about 0.005% to about 0.1%, or about 0.01% to about 0.1%, by weight relative to benzgalantamine of Formula I or a pharmaceutically acceptable salt, or a hydrate thereof, when measured by HPLC.

The composition of the present invention is found to be stable for at least 6 months, and wherein each of the compounds of Formulae A, B, C, or D is present in an amount of about 0.15% or less, by weight, relative to benzgalantamine of Formula I or a pharmaceutically acceptable salt or a hydrate thereof, when measured by HPLC, when the composition is stored for at least 6 months at 40° C.±2° C. temperature and 75±5% relative humidity, or at 25° C.±2° C. temperature and 60±5% relative humidity.

The composition of the present invention further comprises about 0.58 ppm or less of the nitrosamine impurity of compound of Formula E, and about 0.58 ppm or less of the nitrosamine impurity of compound of Formula F,

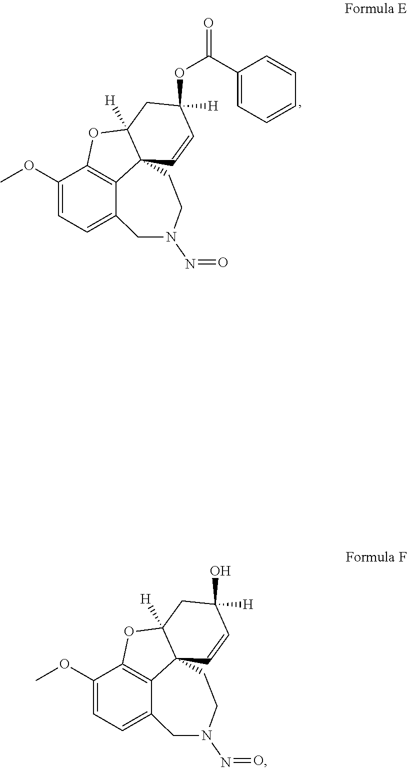 [SMILES: COc1ccc2c3c1O[C@H]1C[C@@H](OC(=O)c4ccccc4)C=C[C@@]31CCN(N=O)C2 ; COc1ccc2c3c1O[C@H]1C[C@@H](O)C=C[C@@]31CCN(N=O)C2]

as determined by an LCMS method.

In another embodiment, the impurity of the compound Formula E, and the compound of Formula F, each present in an amount of about 0.001 ppm to about 0.58 ppm, as determined by LCMS method. For Example, in an amount of about 0.005 ppm to about 0.58 ppm, about 0.006 ppm to about 0.58 ppm, about 0.01 ppm to about 0.58 ppm, about 0.02 ppm to about 0.58 ppm, about 0.01 ppm to about 0.4 ppm, about 0.01 ppm to about 0.3 ppm, about 0.01 ppm to about 0.2 ppm, or about 0.01 ppm to about 0.1 ppm, as determined by an LCMS method.

In another embodiment, the impurity of the compound Formula E, and the compound of Formula F, each present in an amount of about 0.006 ppm to about 0.58 ppm, as determined by LCMS method. In yet another embodiment, the impurity of the compound Formula E, and the compound of Formula F, each present in an amount of about 0.01 ppm to about 0.58 ppm, as determined by LCMS method.

In another embodiment, the impurity of the compound Formula E is present in the composition of the present invention in an amount of about 0.58 ppm or less, for example, in amount of about 0.50 ppm or less, about 0.40 ppm or less, about 0.30 ppm or less, about 0.20 ppm or less, about 0.10 ppm or less, about 0.05 ppm or less, about 0.04 ppm or less, about 0.03 ppm or less, about 0.02 ppm or less, about 0.01 ppm or less, or in a non-detectable amount, as determined by LCMS method.

In another embodiment, the impurity of the compound Formula F is present in the composition of the present invention in an amount of about 0.58 ppm or less, for example, in amount of about 0.50 ppm or less, about 0.40 ppm or less, about 0.30 ppm or less, about 0.20 ppm or less, about 0.10 ppm or less, about 0.05 ppm or less, about 0.04 ppm or less, about 0.03 ppm or less, about 0.02 ppm or less, about 0.01 ppm or less, or in a non-detectable amount, as determined by LCMS method.

In another general aspect, the present invention provides a crystalline form of benzgalantamine of Formula I, characterized by X-ray powder diffraction pattern having peaks expressed in 2θ at 8.1°, 16.3°, 17.7°, 19.9°, and 20.8°±0.2°, when measured using CuKα source.

In another embodiment, the crystalline Form of benzgalantamine of Formula I is further characterized by X-ray powder diffraction pattern having peaks expressed in 2θ at 8.1°, 13.0°, 16.3°, 16.6°, 17.7°, 19.9°, and 20.8°±0.2°, when measured using CuKα source.

In another embodiment, the crystalline Form of benzgalantamine of Formula I is further characterized by X-ray powder diffraction pattern having peaks expressed in 2θ at 8.1°, 11.1°, 13.0°, 13.8°, 16.3°, 16.6°, 17.7°, 18.7, 19.9°, 20.8°, 23.2° and 26.3°±0.2°, when measured using CuKα source.

In another embodiment, the crystalline form of benzgalantamine is further characterized by a differential scanning calorimetry having onset at about 103° C.±5° C. and endothermic peak at about 105° C.±5° C., wherein differential scanning calorimetry analysis is performed at a heating rate of 10° C./minute.

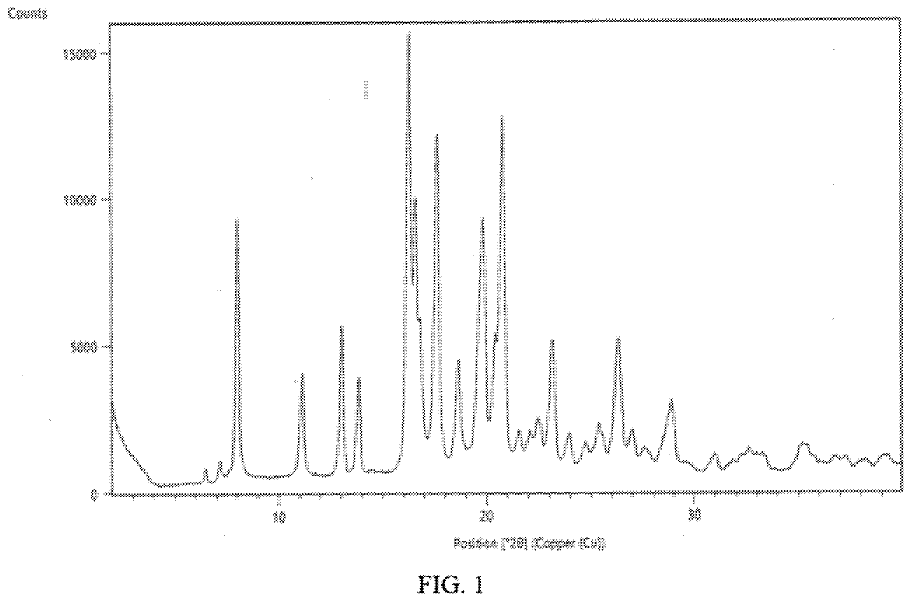

In another embodiment, the crystalline form of benzgalantamine is characterized by X-ray powder diffraction pattern substantially same as depicted in FIG. 1.

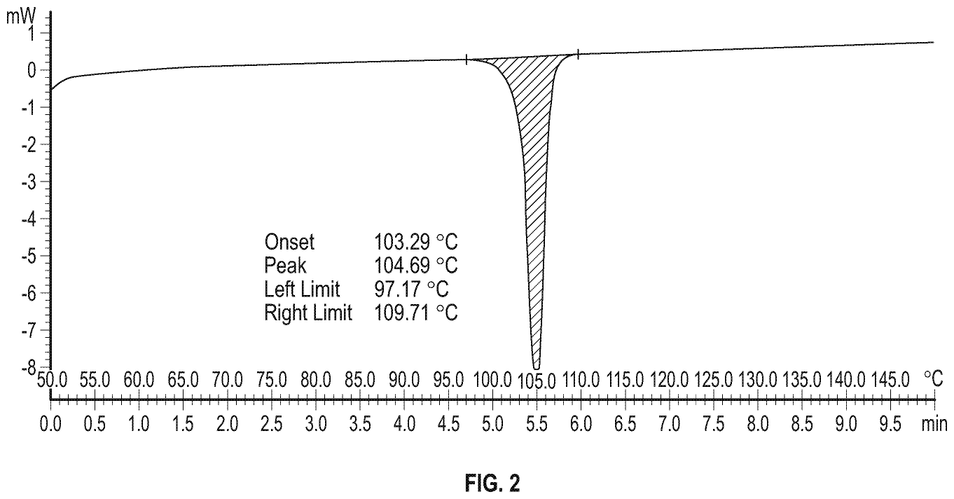

In another embodiment, the crystalline form of benzgalantamine is further characterized by differential scanning calorimetry substantially same as depicted in FIG. 2.

In another embodiment, the crystalline Form of benzgalantamine of Formula I of the present invention is having a purity of about 99% or more, as measured by area percentage of HPLC, and containing one or more impurities of compounds of Formulae A, B, C, and D, wherein each of the compounds of Formulae A, B, C, or D is present in an amount of about 0.15% or less, by weight, relative to benzgalantamine of Formula I, when measured by HPLC, and about 0.58 ppm or less of the nitrosamine impurity of compound of Formula E, and about 0.58 ppm or less of the nitrosamine impurity of compound of Formula F, as determined by an LCMS method.

In another general aspect, the present invention provides a process for the preparation of a crystalline form of benzgalantamine of Formula I, characterized by X-ray powder diffraction pattern having peaks expressed in 2θ at 8.1°, 16.3°, 17.7°, 19.9°, and 20.8°±0.2°, when measured using CuKα source, the process comprising:
(i) adding water to a reaction mass comprising benzgalantamine and acetonitrile, and
(ii) obtaining the crystalline form of benzgalantamine.

In general, the water at step (a) is added at a temperature of about 0° C. to about 10° C.

In general, the reaction mass comprising benzgalantamine and acetonitrile used in step (i) of the above process, can be directly obtained in-situ from a reaction system involving preparation of benzgalantamine wherein acetonitrile is used as one of the solvent.

In general, the crystalline form of benzgalantamine as per step (ii) can be obtained by a process comprising filtration, centrifugation, distillation, evaporation, evaporation under reduced pressure, etc.

In another general aspect, the present invention provides a pharmaceutical composition comprising benzgalantamine or a pharmaceutically acceptable salt thereof substantially free of one or more impurities of compounds of Formulae A, B, C or D, and one or more pharmaceutically acceptable excipients.

In another general aspect, the present invention provides a pharmaceutical composition comprising a composition comprising benzgalantamine of Formula I or a pharmaceutically acceptable salt, or a hydrate thereof, having a purity of about 99% or more, as measured by area percentage of HPLC, and one or more impurities of compounds of Formulae A, B, C, and D, wherein each of the compounds of Formulae A, B, C, or D is present in an amount of about 0.15% or less, by weight, relative to benzgalantamine of Formula I or a pharmaceutically acceptable salt, or a hydrate thereof, when measured by HPLC, and about 0.58 ppm or less of the nitrosamine impurity of compound of Formula E, and about 0.58 ppm or less of the nitrosamine impurity of compound of Formula F; together with one or more pharmaceutically acceptable excipients.

The present invention is further illustrated by the following examples which is provided merely to be exemplary of the invention and do not limit the scope of the invention. Certain modifications and equivalents will be apparent to those skilled in the art and are intended to be included within the scope of the present invention. The examples are set forth to aid in understanding the invention but are not intended to, and should not be construed to limit its scope in any way. The examples do not include detailed descriptions of conventional methods. Such methods are well known to those of ordinary skill in the art and are described in various publications.

### EXAMPLES

The X-ray powder diffraction spectrum was measured under the following experimental conditions:
Instrument: X-Ray Diffractometer, Empyrean, Make: Malvern PANalytical.
X-Ray: Cu K alpha radiation, Tension: 45 KV, Current: 40 mA, Divergence slit: Automatic
Incident beam side: Off set: 0.000, Anti-scatter slit: ½°, Receiving slit: None, Detector: PIXcel1D-Medipix3, Mode: Scanning line detector (1D)
Method parameter: Start position: 2° 2θ, End position: 40° 2θ, Step size: 0.02° rad, Time per step: 67.575s, Scan mode: Continuous

Differential Scanning calorimetry (DSC) analysis was performed using a Differential Scanning calorimeter using Model: DSC 3 Star e, Make: Mettler Toledo, Method: 50° C. to 200° C., Heating rate: 10° C. per minute, Nitrogen flow: 20 mL/min. About 1 to 3 mg of sample was placed at the pan and sealed with the sealing press. The instrument was programmed to heat at a heating rate of 10° C./min in the temperature range between 50° C. to 200° C.

### HPLC Method of Analysis

The purity of benzgalantamine or a pharmaceutically acceptable salt or a hydrate thereof and the content of impurities were determined by following HPLC method of analysis.

HPLC analysis was carried out by using Waters HPLC module e2695 equipped with PDA detector with gradient program and using octadecylsilane HPLC column with column ID (250×4.6) mm, 5 μm.

Mobile phase A was prepared by using buffer (30 mM of di-ammonium hydrogen phosphate and 2 g of hexane-1-sulfonic acid sodium salt and pH adjusted to 6.5 with orthophosphoric acid) and Acetonitrile in the ratio of 95:5 v/v.

Mobile phase B was prepared by using mixture of Acetonitrile and water in the ratio of 80:20 v/v. Detection done at 215 nm at 2.0 ml/min flow rate, column oven temperature 45° C. and 30 μl injection volume.

Gradient program used as below.

Time (min)/% mobile phase B: 0/5, 5/5, 25/30, 40/40, 80/90, 100/90, 101/5 and 120/5.

### Method of Analysis for Nitrosamine Impurities of Compounds of Formulae E and F

The content of nitrosamine impurities of compounds of Formulae E and F, were determined using following LCMS method.

### CHROMATOGRAPHIC CONDITIONS

Equipment: HPLC system equipped with MS detector; Column: Kromasil C18 (150 mm×4.6 mm, 5.0 μm); Wavelength: NA; Flow Rate: 0.7 mL/min; Column temp.: 40° C.; Sampler cooler: 10° C.; Injection Volume: 5 μL; Run time: 20 Minutes

### GRADIENT PROGRAMME

| Time | Mobile phase-A | Mobile phase-B |
|---|---|---|
| 0 | 50 | 50 |
| 5 | 50 | 50 |
| 9 | 20 | 80 |
| 14 | 20 | 80 |
| 15 | 50 | 50 |
| 20 | 50 | 50 |

Diluent: water: methanol=50:50% v/v
Mass parameter: Equipment: Q-trap 6500+ (AB Sciex); Scan type: MRM; Source: ESI
Preparation of Mobile phase A: 10 mM of ammonium acetate in water, pH was adjusted to 4.5 with dilute acetic acid solution.
Preparation of Mobile phase B: Methanol was used as such.
Blank preparation: Diluent was used as blank.

### ABBREVIATIONS

HPLC: High performance liquid chromatography
LOD: Limit of detection
LOQ: Limit of quantification
RH: Relative Humidity

### Example 1: Preparation of Benzgalantamine Using Acetonitrile

Galantamine hydrobromide (150 μm; 0.407 mole) is taken into reaction flask with acetonitrile (525 mL) and charged triethyl amine (123.7 g; 1.22 mole) & dimethyl aminopyridine (49.8 g; 0.407 mole) under stirring. Reaction mass is cooled around 0-10° C. and a solution of benzoyl chloride (63 g; 0.45 mole) & acetonitrile (75 mL) were added dropwise between 0-20° C. Reaction mass is stirred for 1H and monitored by HPLC for completion of reaction. After completion of reaction, water (3000 mL) was added dropwise below 15° C. to precipitate out the product. Slurry was stirred for 1 hour at 10-15° C. & filtered under suction followed by water washing. Drying of the wet cake at 60-70° C. under vacuum afforded the benzgalantamine with 96% yield and with >99.8% HPLC purity.

X-ray powder diffraction pattern is as set forth in FIG. 1.

DSC is as set forth in FIG. 2.

### Example 2: Preparation of Benzgalantamine Using Acetonitrile

Galantamine hydrobromide (110 g, 0.3 mole) is charged into reaction flask along with acetonitrile (385 mL). Triethyl amine (90.7 g; 0.9 mole), 4-dimethylamino pyridine (36.5 g; 0.3 mole) and butylated hydroxy toluene (165 mg; 0.15% w/w) was charged under stirring at ambient temperature. Reaction mass was cooled to 0° C. to 20° C. and a solution of benzoyl chloride (46.2 g; 0.33 Mole) & acetonitrile (55 mL) was added at same temperature. Reaction mass is stirred for 1 hour and the progress of reaction was monitored by HPLC. After completion of reaction, water (2200 mL) was added dropwise along with benzgalantamine seed (0.22 g; 0.2% w/w) at 0° C. to 15° C. to precipitate out the product. Slurry was stirred for 1 hour at 10-15° C. & filtered under suction followed by water washing (220 mL×2). Drying of the wet cake at 60-70° C. under vacuum afforded the benzgalantamine with 96% yield.
HPLC purity: >99.85%.
X-ray powder diffraction pattern is as set forth in FIG. 1.
DSC is as set forth in FIG. 2.
Content of compound of Formula A: 0.02% w/w
Content of compound of Formula B: 0.02% w/w
Content of compound of Formula C: 0.02% w/w
Content of compound of Formula D: 0.04% w/w.

### Example 3: Preparation of Benzgalantamine Gluconate

Benzgalantamine (100 g; 0.255 mole) is charged into reaction flask along with 95% ethanol solution having 5% w/v water (800 mL) under stirring at ambient temperature. D-gluconic acid 8-lactone (82 g; 0.46 mole) was charged and reaction mass was heated to 55-65° C. for 30 minutes. Fine filtration of reaction mass & washing with 95% ethanol solution having 5% w/v water (200 mL) followed by cooling to 25-35° C., seeding with benzgalantamine gluconate (0.5 g; 0.5% w/w) and stirring at 25-35° C. for 46-48 hours precipitated the product. Filtration of product followed by washing with 95% ethanol having 5% w/v water (200 mL×2) afforded the wet cake, which was further stirred in ethanol (800 mL) for 1 hour. Filtration of product under suction followed by washing with ethanol (100 mL×2) and drying at 35 to 45° C. afforded the benzgalantamine gluconate with 80% yield.

TABLE 1
SUMMARY OF HPLC ANALYSIS OF BENZGALANTAMINE
GLUCONATE HPLC purity: ≥99.85%.

| Impurities | Results | LOD | LOQ |
|---|---|---|---|
| Formula A (% w/w) | 0.02 | 0.01 | 0.04 |
| Formula B (% w/w) | 0.03 | 0.01 | 0.05 |
| Formula C (% w/w) | 0.03 | 0.01 | 0.04 |
| Formula D (% w/w) | 0.01 | 0.01 | 0.04 |
| Formula E (ppm) | 0.010 | 0.013 | 0.040 |
| Formula F (ppm) | ND | 0.013 | 0.040 |

N.D. indicates “Not Detected”
LOD indicates “Limit of Detection”
LOQ indicates “Limit of Quantification”

TABLE 3
STABILITY DATA AT 40° C. ± 2° C. (75% ± 5% RH)

| Sr. | | | 1 | 2 | 3 | 6 |
|---|---|---|---|---|---|---|
| No | Tests | Initial | Month | months | months | months |
| 1 | Benzgalantamine | 99.85% | 99.91% | 99.91% | 99.93% | 99.94% |
| | Gluconate | | | | | |
| | (HPLC purity) | | | | | |

Related substance by HPLC (% w/w)

| | | | | | | |
|---|---|---|---|---|---|---|
| 2 | Formula A (% w/w) | 0.02 | 0.03 | 0.02 | 0.03 | 0.02 |
| 3 | Formula B (% w/w) | 0.03 | 0.02 | 0.02 | 0.02 | 0.03 |
| 4 | Formula C (% w/w) | 0.03 | 0.03 | 0.08 | 0.03 | 0.04 |
| 5 | Formula D (% w/w) | 0.01 | 0.01 | 0.03 | 0.01 | 0.01 |

TABLE 4
STABILITY DATA AT 25° C. ± 2° C. (60% ± 5% RH)

| Sr. No | Tests | Initial | 3 Months | 6 Months |
|---|---|---|---|---|
| 1 | Benzgalantamine | 99.85% | 99.94% | 99.95% |
| | Gluconate | | | |
| | (HPLC Purity) | | | |

Related substance by HPLC (% w/w)

| | | | | |
|---|---|---|---|---|
| 2 | Formula A (% w/w) | 0.02 | 0.02 | 0.02 |
| 3 | Formula B (% w/w) | 0.03 | 0.02 | 0.02 |
| 4 | Formula C (% w/w) | 0.03 | 0.03 | 0.03 |
| 5 | Formula D (% w/w) | 0.01 | 0.01 | 0.01 |

### Example 4: Preparation of Benzgalantamine Using Ethylacetate as Solvent

Galantamine hydrobromide (250 mg, 68 mmole) was charged into reaction flask along with ethylacetate (10 mL) followed by triethyl amine (275 mg; 2.71 mmole) and 4-dimethylamino pyridine (83 mg; 68 mmole) under stirring at ambient temperature. Benzoyl chloride (190.85 mg; 135.7 mmole) was added at 25-35° C. temperature. Reaction mass was stirred for 6 hours and the progress of reaction was monitored by HPLC. Distillation of ethylacetate followed by quenching the reaction with water afforded the benzgalantamine.

### Example 5: Preparation of Benzgalantamine Using DMF

Galantamine hydrobromide (250 mg, 68 mmole) is charged into reaction flask along with N,N-dimethyl formamide (4 mL) followed by triethyl amine (275 mg; 2.71 mmole) and 4-dimethylaminopyridine (83 mg; 68 mmole) was charged under stirring at ambient temperature. Benzoyl chloride (190.85 mg; 135.7 mmole) was added at 25-35° C. temperature. Reaction mass was stirred for 24 hours and monitored by HPLC for completion of reaction. Reaction mass was quenched with water to afford the benzgalantamine.

### Example 6: Preparation of Benzgalantamine Using 1,4-Dioxane as Solvent

Galantamine hydrobromide (250 mg, 68 mmole) is charged into reaction flask along with 1,4-dioxane (10 mL) followed by triethyl amine (275 mg; 2.71 mmole) and 4-dimethylaminopyridine (83 mg; 68 mmole) was charged under stirring at ambient temperature. Benzoyl chloride (190.85 mg; 135.7 mmole) was added at 25-35° C. temperature. Reaction mass was stirred for 24 hours and monitored by HPLC for completion of reaction. Reaction mass was quenched with water to afford the benzgalantamine.

### Example 7: Preparation of Benzgalantamine Using Acetone as Solvent

Galantamine hydrobromide (250 mg, 68 mmole) is charged into reaction flask along with acetone (10 mL) followed by triethyl amine (275 mg; 2.71 mmole) and 4-dimethylaminopyridine (83 mg; 68 mmole) was charged under stirring at ambient temperature. Benzoyl chloride (190.85 mg; 135.7 mmole) was added at 25-35° C. temperature. Reaction mass was stirred for 24 hours and monitored by HPLC for completion of reaction. Reaction mass was quenched with water to afford the benzgalantamine.

While the present invention has been described in terms of its specific embodiments, certain modifications and equivalents will be apparent to those skilled in the art and are intended to be included within the scope of the present invention.

1. A process for the preparation of benzgalantamine of Formula I, 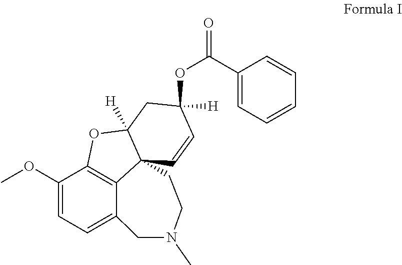 [SMILES: COc1ccc2c3c1O[C@H]1C[C@@H](OC(=O)c4ccccc4)C=C[C@@]31CCN(C)C2]
or a pharmaceutically acceptable salt, or a hydrate thereof, the process comprising:
(a) reacting galantamine or a salt thereof with benzoyl chloride in the presence of one or more bases in one or more solvents to obtain benzgalantamine of Formula I, wherein the solvent is selected from one or more of acetonitrile, acetone, ethylacetate, toluene, N,N-dimethylformamide, tetrahydrofuran, methyl ethyl ketone, and methyl isobutyl ketone; and
(b) optionally, converting the benzgalantamine of Formula I obtained at step (a) to its pharmaceutically acceptable salt, or a hydrate thereof.

2. The process according to claim 1, wherein the base at step (a) is selected from one or more of triethylamine, N,N-diisopropylethylamine, pyridine, and 1,8-diazabicyclo(5.4.0)-undec-7-ene.

3. The process according to claim 1, wherein the reaction at step (a) is performed in the presence of an activating agent 4-dimethylaminopyridine (DMAP).

4. The process according to claim 1, wherein the solvent at step (a) is acetonitrile.

5. The process according to claim 1, wherein the reaction at step (a) is performed optionally in the presence of an anti-oxidant selected from butylated hydroxytoluene (BHT), butylated hydroxyanisole (BHA), sodium thiosulfate, and sodium metabisulfite.

6. The process according to claim 1, wherein the reaction at step (a) is performed at a temperature of about 0° C. to about 50° C.

7. The process according to claim 1, wherein the solvent at step (a) is used in an amount of 2 volumes to 10 volumes of the quantity of galantamine or a salt thereof.

8. The process according to claim 1, wherein the pharmaceutically acceptable salt of benzgalantamine is benzgalantamine gluconate.

9. A compound selected from a compound of Formula A, or a compound of Formula C,

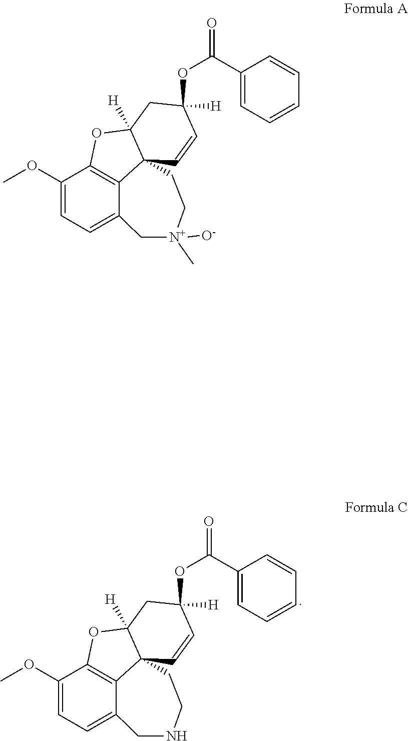

10. A composition comprising benzgalantamine of Formula I, 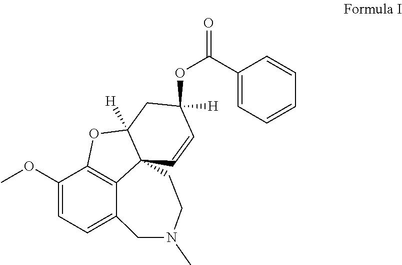 [SMILES: COc1ccc2c3c1O[C@H]1C[C@@H](OC(=O)c4ccccc4)C=C[C@@]31CCN(C)C2]
or a pharmaceutically acceptable salt, or a hydrate thereof, having a purity of about 99% or more, as measured by area percentage of high performance liquid chromatography (HPLC), and one or more impurities of compounds of Formulae A, B, C, and D, wherein each of the compounds of Formulae A, B, C, or D is present in an amount of about 0.15% or less, by weight, relative to benzgalantamine of Formula I or a pharmaceutically acceptable salt, or a hydrate thereof, when measured by HPLC,

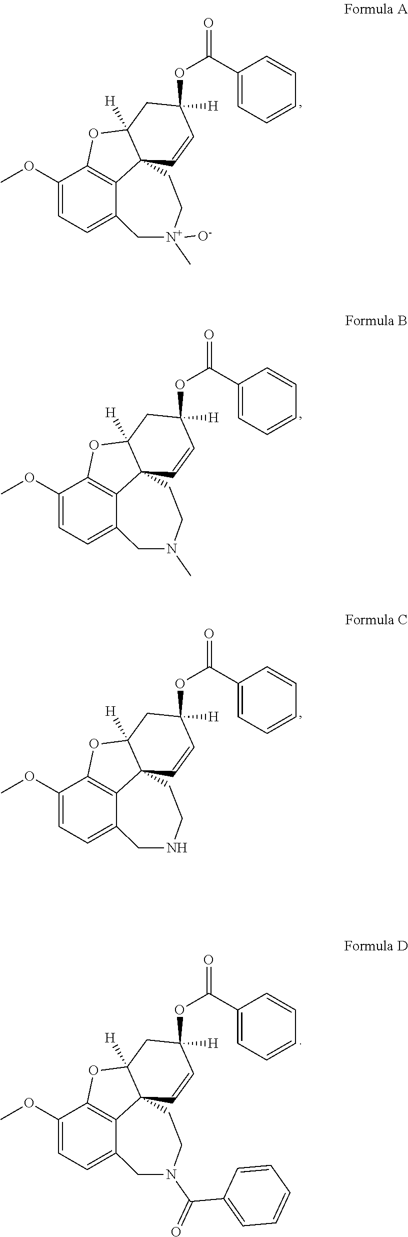

11. The composition according to claim 10, wherein each of the compounds of Formulae A, B, C, or D, when present, is in a detectable amount of about 0.15% or less, by weight, relative to benzgalantamine or a pharmaceutically acceptable salt, or a hydrate thereof, when measured by HPLC.

12. The composition according to claim 10, wherein each of the compounds of Formulae A, B, C, or D, is present in an amount of about 0.003% to about 0.15% by weight, relative to benzgalantamine or a pharmaceutically acceptable salt, or a hydrate thereof, when measured by area percentage of HPLC.

13. The composition according to claim 12, wherein each of the compounds of Formulae A, B, C, or D, is present in an amount of about 0.01% to about 0.15% by weight, relative to benzgalantamine or a pharmaceutically acceptable salt, or a hydrate thereof, when measured by area percentage of HPLC.

14. The composition according to claim 10, wherein the pharmaceutically acceptable salt of benzgalantamine is benzgalantamine gluconate.

15. The composition according to claim 10, wherein the composition is stable for at least 6 months, and wherein each of the compounds of Formulae A, B, C, or D is present in an amount of about 0.15% or less, by weight, relative to benzgalantamine of Formula I or a pharmaceutically acceptable salt, or a hydrate thereof, when measured by HPLC, when the composition is stored for at least 6 months at 40° C.±2° C. temperature and 75±5% relative humidity, or at 25° C.±2° C. temperature and 60±5% relative humidity.

16. The composition according to claim 10, wherein the composition further comprises about 0.58 ppm or less of the impurity of compound of Formula E, and about 0.58 ppm or less of the impurity of compound of Formula F, 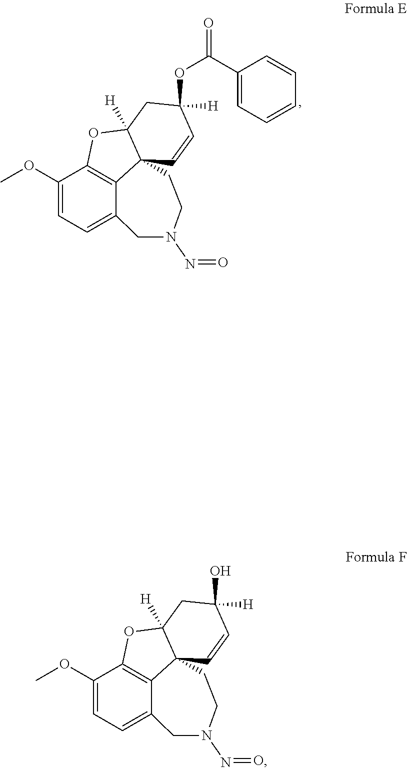 [SMILES: COc1ccc2c3c1O[C@H]1C[C@@H](OC(=O)c4ccccc4)C=C[C@@]31CCN(N=O)C2 ; COc1ccc2c3c1O[C@H]1C[C@@H](O)C=C[C@@]31CCN(N=O)C2]
as determined by an LCMS method.

17. A crystalline Form of benzgalantamine of Formula I, 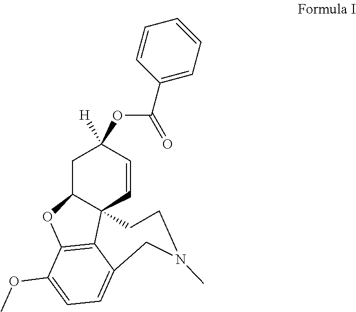
characterized by X-ray powder diffraction pattern having peaks expressed in 20 at 8.1°, 16.3°, 17.7°, 19.9°, and 20.8°±0.2°, when measured using CuKα source.

18. A process for the preparation of a crystalline form of benzgalantamine of Formula I according to claim 17, the process comprising:
(i) adding water to a reaction mass comprising benzgalantamine and acetonitrile, and
(ii) obtaining the crystalline form of benzgalantamine.

19. The process according to claim 18, wherein the water at step (i) is added at a temperature of about 0° C. to about 10° C.

20. A pharmaceutical composition comprising the composition according to claim 16, and one or more pharmaceutically acceptable excipients.
